(19) United States
(12) Patent Application Publication
(10) Pub. No.: US 2006/0085005 A1
(43) Pub. Date: Apr. 20, 2006

(54) Bone drill system with highly visible depth markings

(76) Inventors: James N. Kenealy III, Hobe Sound, FL (US); Zachary B. Suttin, Palm Beach Gardens, FL (US)

Correspondence Address: JENKENS & GILCHRIST, P.C., 225 WEST WASHINGTON, SUITE 2600, CHICAGO, IL, 60606, US

(21) Appl. No.: 10/957,226

(22) Filed: Oct. 1, 2004

(51) Int. Cl. A61B 17/16

(52) U.S. Cl. 606/80

(57) ABSTRACT

A bone drill system, and method for using the same, with a highly visible depth marking for a clinician to more easily visualize the depth marking on a drill bit or similar tool. The drill system comprises a drill body, a drill bit coupled to the drill body, the drill bit having a depth marker measured from a distal end of the drill bit and being adapted to react with ultraviolet light to enhance the visibility of the marker, and an ultraviolet light system coupled to the drill body. The depth marker's reaction with ultraviolet light allows the clinician to more easily visualize the depth marker, thus increasing the accuracy of a depth of a bore made with the drill bit.

### FIELD OF THE INVENTION

This invention relates to devices for developing a bore in bone tissue and, in particular, to highly visible depth markings that allow a clinician to insert a drill bit or other work tool into the bone tissue to a predetermined depth.

### BACKGROUND OF THE INVENTION

It is common for a clinician, such as a dentist, to use tools, such as drills, to create bores in bone tissue of a patient's mouth. Drills come in various styles, sizes, and lengths, but all have a common goal of creating a bore of a known size. The diameter of the drill dictates the diameter of the bore. However, the depth of the bore is determined by the amount of axial movement that the clinician imparts on the drill as he or she inserts it into the bone tissue.

The size of the bore created by the clinician is a critical parameter in the restoration of the dentition of the patient's mouth. For example, if the depth of the bore is too long, it can puncture the sinus cavity if it is placed in the maxilla, or the mandibular canal if it is placed in the mandible. Likewise, the roots of adjacent teeth also can be affected by the size of the bore. Further, if the clinician does not drill to the proper depth, time may be lost to correct the depth of the bore, or failure of the implant to properly integrate into the bone may occur.

To ensure that the drill bit is inserted into the bone to a known depth, the drill bit often contains several markings on it which signify specific depths. For example, FIG. 1 illustrates a typical drill bit 10 used for developing a bore in bone tissue, such as a jawbone. The drill bit 10 includes a drive shank 12 at its proximal end which terminates in a drive attachment 13. The drive attachment 13 is configured to mount with a corresponding tool, such as a dental drill, which imparts rotational motion into the drill bit 10.

The drive shank 12 is connected to a collar 14 that in turn is connected to a plurality of flutes 16. The collar 14 includes a tapered region 15 where it transitions from its larger diameter to the smaller diameter of the flutes 16. Each of the flutes 16 terminates in a cutting edge 18 at a distal end of the drill bit 10. The cutting edge 18 of the flutes 16 slices bone tissue to develop the bore. The number of flutes 16 in the drill bit 10 of FIG. 1 is three, but the number can vary.

Because of the mechanical energy that is converted to heat along the flutes 16 and especially at the cutting edges 18, the drill bit 10 includes an irrigation channel 22 which extends along its length to a point near the cutting edges 18. As seen best in FIG. 3, the irrigation channel 22 terminates in a plurality of openings 24. Accordingly, biocompatible fluid is transported through the irrigation channel 22 and released in the bore via the openings 24 to reduce the friction between the rotating drill bit 10 and the bore tissue. The biocompatible fluid helps inhibit a temperature increase caused by the friction which may harm the bone tissue. As shown, the irrigation channel 22 extends through the drive attachment 13. It is also known in the art that some drill bits do not provide an internal irrigation channel, thus application of biocompatible fluid external to the drill bit may be required.

As is known in the art, the drill bit 10 also includes a plurality of depth markings 26. Each marking 26 signifies a known distance from the cutting edges 18. The clinician drilling the bore uses these depth markings 26 to determine when the distal end of the drill bit 10 has drilled to a known depth. The use of these visual markers is, of course, limited to the clinician's ability to see the mark as the drill bit is being inserted into the patient's mouth. Accordingly, the clinician may have difficulty seeing the mark and the actual bore depth may not correspond to the desired bore depth.

Several types of markings 26 may be used on drill bits including, but not limited to, laser marks, plating materials having contrasting colors, and grooves machined into the drill bits. All of these markers rely on the clinician's ability to see these markers while utilizing the drill bit.

Operating a drill in the oral cavity, or other small closed spaces, is challenging when the target site and the drill are not adequately illuminated. Without proper illumination, the clinician has even more difficulty in seeing the depth marks on a typical dental drill bit, thus, obtaining the proper bore depth is even more challenging. Even though external lighting in a clinical setting is often abundant, the amount of light within the oral cavity is often limited. External light is blocked by the clinician's fingers, hands, arms, and head. Additionally, the drill unit acts as a physical obstruction that may further limit the amount of light within the oral cavity. In addition to objects that prevent light from entering the oral cavity, the accumulation of irrigation fluids, blood, and bone fragments also obscure the clinician's ability to see markers on the drill bit.

Thus, a need exists for an enhanced drill marking system that overcomes the aforementioned problems of known drill marking systems.

### SUMMARY OF THE INVENTION

According to one embodiment of the present invention, a drill system comprises a drill body, a drill bit coupled to the drill body, and an ultraviolet light system coupled to the drill body. The drill body has a handle end and a working end. The drill bit is coupled to the working end of the drill body. The drill bit has a depth marker measured from a distal end of the drill bit. The depth marker is adapted to react with ultraviolet light to enhance the visibility of the marker. The ultraviolet light system applies ultraviolet light to the depth marker.

According to another embodiment of the present invention, a kit of drill bits for producing bores in bone tissue is provided. Each of the drill bits in the kit includes at least one ultraviolet-reactive depth marking that indicates a length from a distal end of the corresponding bit.

According to a further embodiment of the present invention, a drill bit for drilling a bore in bone tissue comprises an elongated body, at least one cutting edge, a plurality of flutes, and a depth marking. The elongated body has a distal end and a proximal end. The proximal end for engaging a rotational-driving device. The cutting edge or edges are at the distal end of the drill bit. The plurality of flutes extend from the distal end toward the proximal end. The plurality of flutes transport bone tissue toward the proximal end. The depth marking is located at a predetermined distance from the distal end of the elongated body. The depth marking includes a fluorescing pigment.

According to yet another embodiment of the present invention, a system for inserting a working tool into living tissue comprises a main body, a tool, and an ultraviolet light system. The main body has a handle end and a working end. The tool has a distal end for insertion into the living tissue. The tool also has a depth marker indicating the distance from the distal end. The depth marker reacts with ultraviolet light to enhance the visibility of the marker. The ultraviolet light system applies ultraviolet light to the depth marker.

According to one method of the present invention, a method for identifying the depth of insertion of a tool used with living tissue is provided. The method includes providing a working tool with a distal end to be inserted into the living tissue. The tool has a depth marker that indicates a distance from the distal end. The depth marker reacts with ultraviolet light to enhance the visibility of the marker. The method further includes applying ultraviolet light on the tool to enhance the visibility of the depth marker.

According to yet another method of the present invention, a method for drilling a bore in living bone is provided. The method provides a drill bit that has a distal end to be inserted into the living bone. The drill bit has a depth marker to indicate the distance from the distal end. The depth marker reacts with ultraviolet light to enhance the visibility of the marker. The method includes using a power-driven mechanism to rotate the drill bit, inserting the distal end of the drill bit into the living bone, and applying ultraviolet light on the drill bit while the drill bit is inserted into living bone to enhance the visibility of the depth marker.

According to yet a further embodiment of the present invention, a drill bit for drilling a bore in bone tissue comprises an elongated body, at least one cutting edge, a plurality of flutes, and a depth marking. The elongated body has a distal end and a proximal end. The proximal end is for engaging a rotational-driving device. The cutting edge or edges are at the distal end of the drill bit. The plurality of flutes extend from the distal end toward the proximal end. The plurality of flutes transport bone tissue toward the proximal end. The depth marking is located at a predetermined distance from the distal end of the elongated body. The depth marking includes a phosphorescing pigment.

According to still yet another embodiment of the present invention, a kit of drill bits for producing bores in bone tissue is provided. Each of the drill bits in the kit includes at least one phosphorescent depth marking that indicates a length from a distal end of the corresponding bit.

According to still yet a further embodiment of the present invention, a system for inserting a working tool into living tissue comprises a main body, a tool, and an ultraviolet light system. The main body has a handle end and a working end. The tool has a distal end for insertion into the living tissue. The tool also has a phosphorescing depth marker indicating the distance from the distal end. The phosphorescing depth marker reacts with ultraviolet light to enhance the visibility of the marker. The ultraviolet light system applies ultraviolet light to the depth marker.

According to yet a further method of the present invention, a method for identifying the depth of insertion of a tool used with living tissue is provided. The method provides a working tool with a distal end to be inserted into the living tissue. The tool has a phosphorescing depth marker that indicates a distance from the distal end. The phosphorescing depth marker reacts with ultraviolet light to enhance the visibility of the marker. The method applies ultraviolet light on the tool to enhance the visibility of the phosphorescing depth marker.

The above summary of the present invention is not intended to represent each embodiment or every aspect of the present invention. The detailed description and Figures will describe many of the embodiments and aspects of the present invention.

### BRIEF DESCRIPTION OF THE DRAWINGS

The foregoing and other advantages of the invention will become apparent upon reading the following detailed description and upon reference to the drawings.

FIG. 1 is a side view of a prior art drill bit for a bone;

FIG. 2 is an end view of the cutting edges of the drill bit in FIG. 1;

FIG. 3 is a cross-sectional view taken through 3-3 in FIG. 1;

FIG. 4 is a side view of a drill system according to one embodiment of the present invention;

FIG. 5 is a side view of a drill body for use in the drill system according to one embodiment of the present invention; and

FIG. 6 is side view of a drill bit for use in the drill system according to one embodiment of the present invention.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE INVENTION

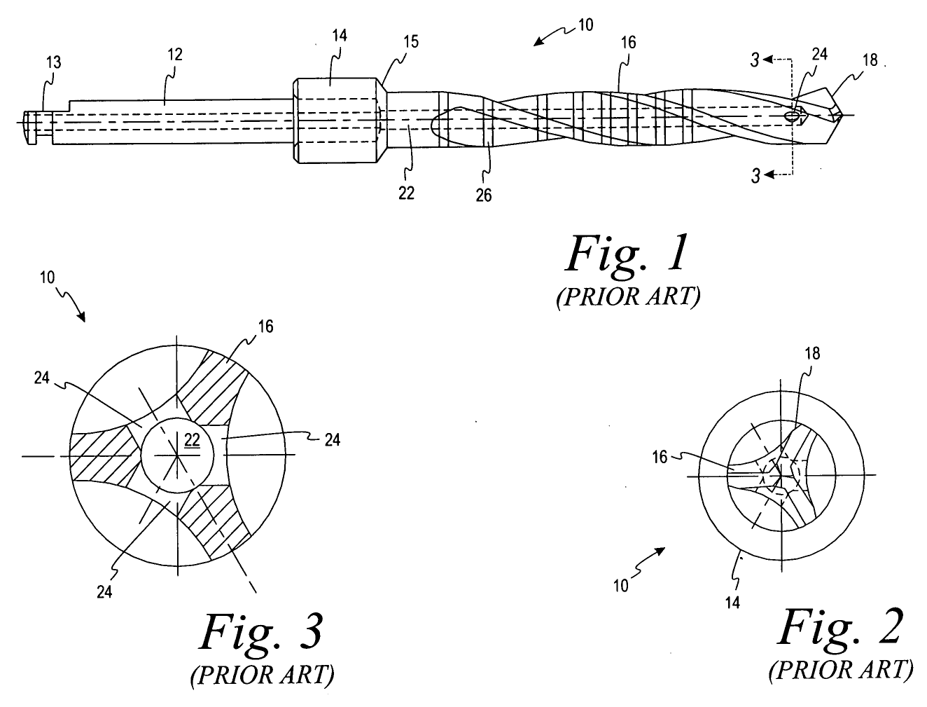

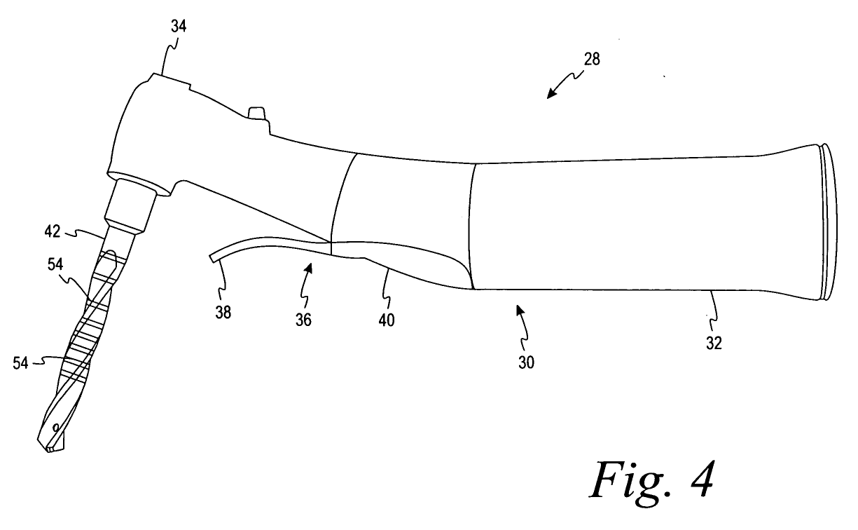

As described in the background section, FIGS. 1-3 describe a prior art drill bit 10. The inventive drill bit and system includes many of the same mechanical features of the drill bit 10, but includes an enhanced marking system that allows exceptional visibility for the clinician. FIG. 4 depicts a drill system 28 for developing a bore in bone tissue, such as a jawbone, featuring highly visible depth markings. The drill system 28 comprises a drill body 30, a drill bit 42, and an ultraviolet light system 36. The drill body 30 comprises a handle end 32 and a working end 34. The drill body 30 may be, for example, a dental handpiece. The handle end 32 is used by a clinician to grasp and operate the drill system 28. The handle end 32 may contain controls, such as switches, to activate and control the drill system 28. The working end 34 of the drill body 30 is adapted to drive a work tool. The work tool is rotationally driven by the working end 34. Examples of working tools that may be used in connection with the drill body 30 include, but are not limited to, drill bits, bone taps creating internal threads in a bore, pilot drill bits, and trephines.

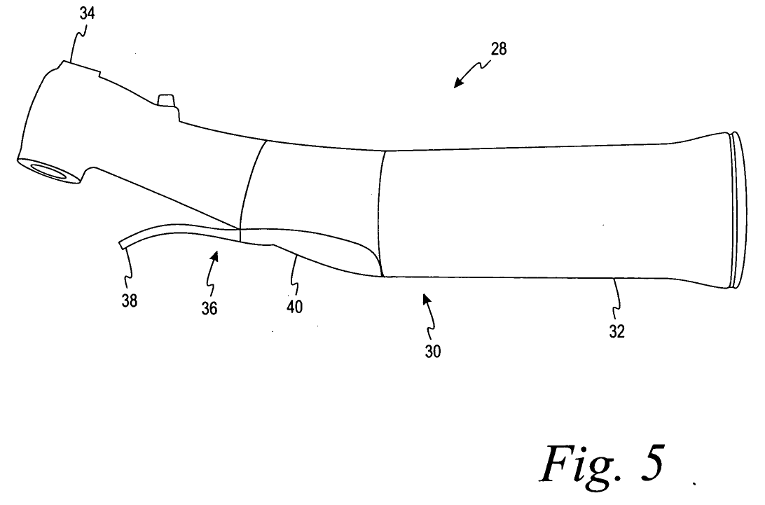

Turning now to FIG. 5, the ultraviolet light system 36 is shown coupled to the drill body 30 to be used in connection with a working tool featuring depth marks. The ultraviolet light system 36 comprises a light-transmitting pipe 38 and an ultraviolet light source 40. The light-transmitting pipe 38 guides ultraviolet light from the source 40 towards the working end 34 of the drill body 30. As one example, the light pipe 38 comprises a polymeric material that is not electrically conductive. The light pipe 38 has a diameter of about 2 mm. According to one embodiment of the present invention, the light pipe 38 is a single-use, disposable light pipe, and will be removed from the ultraviolet light system 36 following its use on one patient. It is further contemplated that the light pipe 38 can be in other forms, such as a fiber-optic cable.

The ultraviolet light source 40 of the ultraviolet light system 36 is an ultraviolet light emitting diode (“UV LED”) according to one embodiment of the present invention. The UV LED is controlled by a circuit board and powered by a battery or other power source. A manifold operatively connects the light pipe 38 to the UV LED of the light source 40. The manifold also establishes a proper orientation between the light pipe 38 and the UV LED of the light source 40 to allow maximum light transmission. According to one embodiment, the light pipe 38 is oriented at an 180° angle to the UV LED of the light source 40 for maximum light transfer.

It is further contemplated that the ultraviolet light system 36 may be either integrally designed as a part of the drill body 30, a separate device that is simply secured to the drill body 30 by retaining clips, or the ultraviolet light system 36 may be a completely separate device located away from the drill body. Many dental power-driven mechanisms, such as drills, are already in existence. Therefore, it is contemplated that a retrofit ultraviolet light kit may be attached to an existing power-driven mechanism to allow a clinician to utilize a power-driven mechanism they already possess. It is contemplated that the ultraviolet light kit may be connected to the power-driven mechanism in a variety of manners including but not limited to, screws and other mechanical fasteners, velcro straps, glue and other adhesives, or retaining clips.

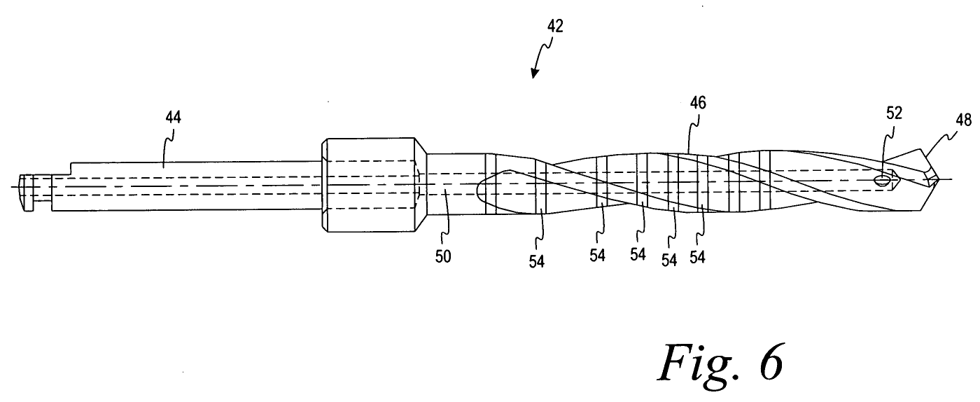

Referring next to FIG. 6, the drill bit 42 featuring highly visible depth markers of the drill system 28 (FIG. 4) is shown. The drill bit 42 is adapted to be inserted into one of a patient's bones, such as the mandible or the maxilla. The drill bit 42 of FIG. 6 is made of stainless steel, although other materials are possible. The drill bit 42 includes a drive shank 44 at its proximal end. The drive shank 44 is configured to mount with a corresponding tool, such as the working end 34 of the drill body 30 (FIG. 4), which imparts rotational motion into the drill bit 42.

The drill bit 42 contains a plurality of flutes 46. Each of the flutes 46 terminates in a cutting edge 48 at a distal end of the drill bit 42. The cutting edge 48 of the flutes 46 slices bone tissue to develop a bore. The number of flutes 46 in the drill bit 42 of FIG. 6 is three, but the number can vary.

Because of the mechanical energy that is converted to heat along the flutes 46 and especially at the cutting edges 48, the drill bit 42 includes an irrigation channel 50 which extends along its length to a point near the cutting edges 48. The irrigation channel 50 terminates in a plurality of openings 52. Accordingly, biocompatible fluid is transported through the irrigation channel 50 and released in the bore via the openings 52 to reduce the friction between the rotating drill bit 42 and the bore tissue.

The drill bit 42 also includes a plurality of depth markings 54. Each marking 54 signifies a known distance from the cutting edges 48. The clinician drilling the bore uses these depth markings 54 to determine when the distal end of the drill bit 42 is at a known depth. According to one embodiment, the depth markings 54 are coated with pigments that react with ultraviolet light (“UV light”) causing fluorescence to occur and enhance the visibility of the markings 54. The pigments are excited by UV light causing fluorescence to occur. Pigments known to react with UV light and fluoresce include, but are not limited to: fluorite, zinc, cadmium, copper ores, willemmite, Rhodamine B, para toluene sulfonamide (PTSA), benzoguanamine, aliphatic diamines, isophthalic acid, polyfunctional glycols, phthalic anhydride, zinc sulfide, zinc cadmium sulfide, salicylic aldazines, and alkali earth metals. The excitation of the pigments causes higher Singlet energy states, which then revert to their original ground state by emitting a portion of their absorbed energy as light. According to one embodiment of the present invention, the pigments are contained in a biocompatible UV-light-sensitive paint that is applied to the drill bit 42 at the depth markings 54. An example of a suitable UV light sensitive paint is WILDFIRE™ paint manufactured by MODERN MASTERS® Inc., of North Hollywood, Calif.

The pigments that react with UV light and fluoresce may be selected so that the markings 54 appear as brightly colored bands during use of the drill bit 42 and provide a great deal of contrast to the rest of the drill bit 42. Thus, the markings 54 are easily visible to the clinician. The markings 54 emit visible light in response to the ultraviolet light and appear to be “glowing.” The fact that the markings 54 emit light, rather than simply reflect light as is the case with prior art systems, allows the clinician to view the markings 54 within the patient's mouth more easily, particularly if the external light is partially blocked by the clinician.

It is additionally contemplated that different pigments may be used for each depth mark 54, such that the color of each depth mark is different when the UV light is applied to excite the pigment and cause fluorescence. Having each depth mark 54 a different color allows the clinician to better differentiate between each depth mark 54.

According to another embodiment, the depth markings 54 are coated with pigments that react with ultraviolet light (“UV light”) causing phosphorescence to occur to enhance the visibility of the markings 54. The pigments are excited by UV light causing phosphorescence to occur. Phosphorescent pigment coated depth markings are similar to the fluorescent pigment coated depth markings, however, the phosphorescent depth markings continue to emit light even when UV light is not being applied. Pigments known to react with UV light and cause phosphorescence to occur include, but are not limited to: strontium aluminate, strontium sulfide, cadmium sulfide, calcium sulfide, zinc sulfide, Benzoxazoles, Willemite, Calcite, Franklinite, Benitoite, Scheelite, Adamite, Fluorite, Sodalite, gypsum, Stilbene triazinies, 4,4′-bis(2-sulphostyryl) biphenyl, riboflavin, ruby, talc, opal, agate, quartz, and amber. The excitation of the pigments causes higher Singlet energy states, which then revert to their original ground state by emitting a portion of their absorbed energy as light over an extended period of time.

The pigments that react with UV light and phosphoresce may be selected so that the markings 54 appear as brightly colored bands during use of the drill bit 42 and provide a great deal of contrast to the rest of the drill bit 42. Thus, the markings 54 are easily visible to the clinician. The markings 54 emit visible light in response to the ultraviolet light and appear to be “glowing.” The fact that the markings 54 emit light, rather than simply reflect light as is the case with prior art systems, allows the clinician to view the markings 54 within the patient's mouth more easily, particularly if the external light is partially blocked by the clinician.

It is additionally contemplated that different pigments may be used for each depth mark 54, such that the color of each depth mark is different when the UV light is applied to excite the pigment and cause phosphorescence. Having each depth mark 54 a different color allows the clinician to better differentiate between each depth mark 54.

The depth marks 54 may either be solid bands around the perimeter of the drill bit 42 or a dot or a series of dots located around the perimeter of the drill bit. The dot or series of dots appear to form a solid line around the perimeter of the drill bit when ultraviolet light is applied to the drill bit 42 as it turns.

According to yet another embodiment of the present invention, a number of depth marks on a drill bit are applied using a UV-light-sensitive paint. After the paint has been applied, an epoxy layer is applied over the UV-light-sensitive paint. The epoxy layer seals the UV-light-sensitive paint, enhancing the ability of the UV-light-sensitive paint to remain on the drill bit while the drill bit is being used and during sterilization of the drill bit. The epoxy layer prevents the UV-light-sensitive paint of the depth marks from coming in direct contact with the bone during use of the drill bit. The pigment could also be encapsulated in an acrylic plug applied to the drill bit.

While the pigment-coated depth marks have been described in use in connection with a drill bit, it is contemplated that pigment-coated depth marks could also be used in connection with other tools. Examples of these tools include, but are not limited to, osteotomes, bone taps that create internal threads in a bore, pilot drill bits, trephines, and other similar surgical tools where a need exists to control the depth of the tool. Exemplary power driven osteotome tools are described in U.S. Pat. No. 6,171,312, which is incorporated by reference herein in its entirety.

The drill bit 42 can be a member of a drill bit set in which each drill bit has the UV-sensitive depth marks that fluoresce or phosphoresce. In a typical drill bit set for an entire product line of dental implants, a supplier may have, for example, drill bits with flute diameters of 2.00 mm, 2.75 mm, 3.00 mm, 3.15 mm, 3.25 mm, 4.25 mm, and 5.25 mm. Each flute diameter (e.g., seven of them) may also may be made in one or multiple lengths.

In operation, the proximal end of the drill bit 42 is inserted into the working end 34 of the drill body 30, as shown in FIG. 4. The UV-sensitive depth markers 54 on the drill bit 42 have UV light applied to enhance the visibility of the markers 54 by causing fluorescence or phosphorescence to occur. The ultraviolet light source 40 generates UV light. The light-transmitting pipe 38 guides the UV light from the light source 40 towards the working end 34 of the drill body 30 and the drill bit 42. The UV light reacts with the pigments on the depth markers 54, enhancing the visibility of the markers by causing fluorescence or phosphorescence to occur. The system supplies power to the working end 34 to rotationally drive the drill bit 42. The clinician then inserts the distal end of the drill bit 42 into living bone of a patient. While the clinician inserts the drill bit 42 into the bone, the UV light continues to react with the markers 54 to enhance the clinician's ability to see the markers 54.

According to an alternate embodiment of the present invention, depth markings on a drill bit are coated with pigments that react with infrared light (“IR light”) to enhance the visibility of the markings. The markings that react with IR light are similar to the pigments that react with UV light previously described, except that the markings are excited by IR light, rather than UV light. The drill bits that have depth markings with pigments that react with IR light also may be provided in kits of various sizes of drill bits.

While the present invention has been described with reference to a dental drill, it is also contemplated that the present invention may be used with orthopedic drills and orthopedic tools.

It is further contemplated that the present invention may be used with any drill or rotational tool, not limited to dental or orthopedic applications, but including, for example, carpentry tools, masonry tools, and metal working tools.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the claimed invention, which is set forth in the following claims.

1. A drill system for developing a bore in bone, comprising:
a drill body having a handle end and a working end;
a drill bit coupled to the working end of the drill body and for insertion into the bone, the drill bit having a depth marker measured from a distal end of the drill bit, the depth marker being adapted to react with ultraviolet light to enhance the visibility of the marker; and
an ultraviolet light system coupled to the drill body and applying ultraviolet light to the depth marker.

2. The drill system of claim 1, wherein the ultraviolet light system includes a light-transmitting pipe for transmitting the ultraviolet light toward the drill bit.

3. The drill system of claim 2, wherein the ultraviolet light system includes an ultraviolet light source for generating the ultraviolet light for receipt by the light-transmitting pipe.

4. The drill system of claim 3, wherein the ultraviolet light source is an ultraviolet light emitting diode.

5. The drill system of claim 2, wherein the light-transmitting pipe is a fiber-optic cable.

6. The drill system of claim 1, wherein the ultraviolet light system includes an ultraviolet light source for generating the ultraviolet light.

7. The drill system of claim 6, wherein the ultraviolet light source is an ultraviolet light emitting diode.

8. The drill system of claim 1, further including a plurality of depth markings measured from the distal end of the drill bit and being adapted to react with ultraviolet light to enhance the visibility of the markers.

9. The drill system of claim 8, wherein each of the plurality of markings is a different color when subjected to the ultraviolet light.

10. The drill system of claim 1, wherein the marker includes fluorite.

11. The drill system of claim 1, wherein the marker includes willemmite.

12. The drill system of claim 1, wherein the marker includes para toluene sulfonamide (PTSA).

13. The drill system of claim 1, wherein the marker includes benzoguanamine.

14. The drill system of claim 1, wherein the marker includes aliphatic diamines and isophthalic acid.

15. The drill system of claim 1, wherein the marker includes polyfunctional glycols and phthalic anhydride.

16. The drill system of claim 1, wherein the ultraviolet light system is integrally connected to the drill body.

17. The drill system of claim 1, wherein the ultraviolet light system is connected to the drill body using at least one retaining clip.

18. The drill system of claim 1, wherein the drill body is a dental handpiece.

19. The drill system of claim 1, wherein the marker is a plurality of dots around a perimeter of the drill bit.

20. The drill system of claim 1, wherein the marker is a solid band around a perimeter of the drill bit.

21. The drill system of claim 1, wherein the marker is a dot on the drill bit.

22. The drill system of claim 1, wherein the marker includes an epoxy cover.

23. The drill system of claim 1, wherein the marker fluoresces to enhance the visibility of the marker.

24. The drill system of claim 1, wherein the marker phosphoresces to enhance the visibility of the marker.

25. A kit of drill bits for producing bores in bone tissue, each of the drill bits in the kit include at least one ultraviolet-reactive depth marking that indicates a length from a distal end of the corresponding bit.

26. The kit of drill bits of claim 25, wherein the marking includes fluorite.

27. The kit of drill bits of claim 25, wherein the marking includes willemmite.

28. The kit of drill bits of claim 25, wherein the marking includes para toluene sulfonamide (PTSA).

29. The kit of drill bits of claim 25, wherein the marking includes benzoguanamine.

30. The kit of drill bits of claim 25, wherein the marking includes aliphatic diamines and isophthalic acid.

31. The kit of drill bits of claim 25, wherein the marking includes polyfunctional glycols and phthalic anhydride.

32. The kit of drill bits of claim 25, wherein the marking has a color indicative of the length from a distal end of the bit.

33. The kit of drill bits of claim 25, wherein the marking is a plurality of dots around a perimeter of each of the corresponding drill bits.

34. The kit of drill bits of claim 25, wherein the marking is a solid band around a perimeter of each of the corresponding drill bits.

35. The kit of drill bits of claim 25, wherein the marking includes an epoxy cover.

36. A system for inserting a working tool into living tissue, comprising:
a main body having a handle end and a working end;
a tool having a distal end for insertion into the living tissue, the tool having a fluorescing depth marker indicating a distance from the distal end, the fluorescing depth marker being reacted with ultraviolet light to enhance the visibility of the marker; and
an ultraviolet light system for applying ultraviolet light on the depth marker.

37. The system of claim 36, wherein the tool is a drill bit.

38. The system of claim 36, wherein the tool is an osteotome.

39. The system of claim 36, wherein the tool is a bone tap for creating threads in a bore.

40. The system of claim 36, wherein the tool is a pilot drill bit.

41. The system of claim 36, wherein the tool is a trephine.

42. The system of claim 36, wherein the tool is a countersink drill bit.

43. The system of claim 36, wherein the tool is a shaping drill bit.

44. The system of claim 36, wherein the tool is a bone profiler drill bit.

45. The system of claim 36, wherein the ultraviolet light system includes a light-transmitting pipe.

46. The system of claim 36, wherein the ultraviolet light system includes an ultraviolet light source for generating ultraviolet light.

47. The system of claim 46, wherein the ultraviolet light source is an ultraviolet light emitting diode.

48. The system of claim 36, further including a plurality of markings measured from the distal end of the tool and being adapted to react with ultraviolet light to enhance the visibility of the markers.

49. A method for identifying the depth of insertion of a tool used with living tissue, comprising:
providing a working tool having a distal end to be inserted into the living tissue, the tool having a fluorescing depth marker indicating a distance from the distal end, the fluorescing depth marker being reactable with ultraviolet light to enhance the visibility of the marker; and
applying ultraviolet light on the tool to enhance the visibility of the fluorescing depth marker.

50. The method of claim 49, further including inserting the working tool into the living bone.

51. The method of claim 49, wherein the applying includes passing ultraviolet light through a light-transmitting pipe.

52. The method of claim 51, wherein the light-transmitting pipe is located on a driving mechanism for driving the tool.

53. The method of claim 49, wherein the applying includes generating ultraviolet light with an ultraviolet light source.

54. The method of claim 53, wherein the generating ultraviolet light with the ultraviolet light source includes integrally attaching the ultraviolet light source to a driving mechanism for the working tool.

55. The method of claim 53, wherein the generating ultraviolet light with the ultraviolet light source includes attaching the ultraviolet light source to a driving mechanism for the working tool using a retaining clip.

56. A method for drilling a bore in living bone, comprising:
providing a drill bit having a distal end to be inserted into the living bone, the drill bit having a depth marker indicating a distance from the distal end, the depth marker being made of a material reactable with ultraviolet light to enhance the visibility of the marker;
using a power-driven mechanism to rotate the drill bit;
inserting the distal end into the living bone; and
while the inserting occurs, applying ultraviolet light on the drill bit to enhance the visibility of the depth marker.

57. The method of claim 56, wherein the depth marker material is selected from the group consisting of fluorite, willemmite, para toluene sulfonamide (PTSA), benzoguanamine, aliphatic diamines, isophthalic acid, polyfunctional glycols, and phthalic anhydride.

58. The method of claim 56, wherein the depth marking material is selected from the group consisting of strontium aluminate, dtomtium sulfide, cadmium sulfide, calcium sulfide, zinc sulfide, benzoxazoles, willemite, calcite, franklinite, benitoite, scheelite, adamite, fluorite, sodalite, gypsum, stilbene triazines, 4,4′-bis(2-sulphostyryl) biphenyl, and riboflavin.

59. A kit of drill bits for producing bores in bone tissue, each of the drill bits in the kit including at least one infrared-reactive depth marking that indicates a length from the distal end of the corresponding bit.

60. A drill bit for drilling a bore in bone tissue, comprising:
an elongated body having a distal end and a proximal end, the proximal end for engaging a rotational-driving device;
at least one cutting edge at the distal end;
a plurality of flutes that extend from the distal end toward the proximal end, the plurality of flutes providing a passageway for transporting bone tissue toward the proximal end; and
a depth marking located on the elongated body at a predetermined distance from the distal end, the depth marking including a phosphorescing pigment.

61. The drill bit of claim 60, wherein the depth marking further includes epoxy applied over the phosphorescing pigment.

62. The drill bit of claim 60, further comprising a plurality of depth markings, wherein each of the plurality of markings includes a different colored phosphorescing pigment.

63. The drill bit of claim 60, wherein the depth marking is a single dot located on the drill bit.

64. The drill bit of claim 60, wherein the phosphorescing pigment is excited by ultraviolet light.

65. The drill bit of claim 60, wherein the phosphorescing pigment is excited by infrared light.

66. The drill bit of claim 60, wherein the phosphorescing pigment is selected from the group consisting of strontium aluminate, dtomtium sulfide, cadmium sulfide, calcium sulfide, zinc sulfide, benzoxazoles, willemite, calcite, franklinite, benitoite, scheelite, adamite, fluorite, sodalite, gypsum, stilbene triazines, 4,4′-bis(2-sulphostyryl) biphenyl, and riboflavin.

67. A kit of drill bits for producing bores in bone tissue, each of the drill bits in the kit include at least one phosphorescent depth marking that indicates a length from a distal end of the corresponding bit.

68. The kit of drill bits of claim 67, wherein the at least one marking has a color indicative of the length from a distal end of the bit.

69. The kit of drill bits of claim 67, wherein the at least one marking is a plurality of dots around a perimeter of each the corresponding drill bits.

70. The kit of drill bits of claim 67, wherein the at least one marking is a solid band around a perimeter of each of the corresponding drill bits.

71. The kit of drill bits of claim 67, wherein the at least one marking includes an epoxy cover.

72. The kit of drill bits of claim 67, wherein the at least one marking is a single dot on a perimeter of each of the corresponding drill bits.

73. The kit of drill bits of claim 67, wherein the at least one phosphorescent depth marking is an ultraviolet reactive phosphorescent depth marking.

74. The kit of drill bits of claim 67, wherein the at least one phosphorescent depth marking is an infrared reactive phosphorescent depth marking.

75. A system for inserting a working tool into living tissue, comprising:
a main body having a handle end and a working end;
a tool having a distal end for insertion into the living tissue, the tool having a phosphorescing depth marker indicating a distance from the distal end, the phosphorescing depth marker being reacted with ultraviolet light to enhance the visibility of the marker; and
an ultraviolet light system for applying ultraviolet light on the depth marker.

76. The system of claim 75, wherein the tool is a drill bit.

77. The system of claim 75, wherein the tool is a osteotome.

78. The system of claim 75, wherein the tool is a bone tap for creating threads in a bore.

79. The system of claim 75, wherein the tool is a pilot drill bit.

80. The system of claim 75, wherein the tool is a trephine.

81. The system of claim 75, wherein the tool is a countersink drill bit.

82. The system of claim 75, wherein the tool is a shaping drill bit.

83. The system of claim 75, wherein the tool is a bone profiler drill bit.

84. The system of claim 75, wherein the ultraviolet light system includes a light-transmitting pipe.

85. The system of claim 75, wherein the ultraviolet light system includes an ultraviolet light source for generating ultraviolet light.

86. The system of claim 75, wherein the ultraviolet light source is an ultraviolet light emitting diode.

87. The system of claim 75, further including a plurality of phosphorescing depth markers measured from the distal end of the tool and being adapted to react with ultraviolet light to enhance the visibility of the markers.

88. A method for identifying the depth of insertion of a tool used with living tissue, comprising:
providing a working tool having a distal end to be inserted into the living tissue, the tool having a phosphorescing depth marker indicating a distance from the distal end, the phosphorescing depth marker being reactable with ultraviolet light to enhance the visibility of the marker; and
applying ultraviolet light on the tool to enhance the visibility of the phosphorescing depth marker.

89. The method of claim 88, further including inserting the working tool into the living bone.

90. The method of claim 88, wherein the applying includes passing ultraviolet light through a light-transmitting pipe.

91. The method of claim 90, wherein the light-transmitting pipe is located on a driving mechanism for driving the tool.

92. The method of claim 88, wherein the applying includes generating ultraviolet light with an ultraviolet light source.

93. The method of claim 92, wherein the generating ultraviolet light with the ultraviolet light source includes integrally attaching the ultraviolet light source to a driving mechanism for the working tool.

94. The method of claim 92, wherein the generating ultraviolet light with the ultraviolet light source includes attaching the ultraviolet light source to a driving mechanism for the working tool using a retaining clip.

95. A drill bit for drilling a bore in bone tissue, comprising:
an elongated body having a distal end and a proximal end, the proximal end for engaging a rotational-driving device;
at least one cutting edge at the distal end;
a plurality of flutes that extend from the distal end toward the proximal end, the plurality of flutes providing a passageway for transporting bone tissue toward the proximal end; and
a depth marking located on the elongated body at a predetermined distance from the distal end, the depth marking including a fluorescing pigment.

96. The drill bit of claim 95, wherein the depth marking further includes epoxy applied over the fluorescing pigment.

97. The drill bit of claim 95, further comprising a plurality of depth markings, wherein each of the plurality of markings includes a different colored fluorescing pigment.

98. The drill bit of claim 95, wherein the depth marking is a single dot located on the drill bit.

99. The drill bit of claim 95, wherein the fluorescing pigment is excited by ultraviolet light.

100. The drill bit of claim 95, wherein the fluorescing pigment is excited by infrared light.

101. The drill bit of claim 95, wherein the fluorescing pigment is selected from the group consisting of fluorite, willemmite, para toluene sulfonamide (PTSA), benzoguanamine, aliphatic diamines, isophthalic acid, polyfunctional glycols, and phthalic anhydride.
